(19) United States
(12) Patent Application Publication
(10) Pub. No.: US 2026/0192109 A1
(43) Pub. Date: Jul. 9, 2026

(54) METHOD FOR ADAPTING MICRONEEDLING APPARATUS FOR INTRACUTANEOUS NEUROPATHY INTERVENTIONS

(71) Applicant: GLOBAL MEDICAL INSTITUTE SA, Lausanne (CH)

(72) Inventors: Giorgio Pietramaggiori, Saint-Sulpice (CH); Sandra Scherer-Pietramaggiori, Saint-Sulpice (CH); Giovanni Riva, Modena (IT)

(21) Appl. No.: 18/868,363

(22) PCT Filed: May 22, 2023
(86) PCT No.: PCT/IB2023/055234

§ 371 Date: Nov. 22, 2024

(30) Foreign Application Priority Data
May 24, 2022 (CH) 00624/2022
Nov. 8, 2022 (CH) 01331/2022

(51) Int. Cl. A61N 1/36

(52) U.S. Cl. CPC A61N 1/36021; A61N 1/36017; A61N 1/36031

(57) ABSTRACT

The invention concerns a method for controlling and/or adapting a microneedling apparatus, having one or more microneedles and means to provide radiofrequency (RF) energy, for treatment of dysfunctions of intracutaneous nerve fibers and/or the neurovascular plexus, comprising setting a defined percutaneous penetration depth for the one or more microneedles ranging from 0.5 mm to 3.5 mm, and setting an RF energy output value of 5 mJ to 60 mJ per needle, preferably of 10 mJ to 40 mJ per needle.

### TECHNICAL DOMAIN

The present invention concerns a method to parametrize a microneedling system for treatment of dysfunctions of intracutaneous nerve fibers and/or blood vessels, including in the management of headache disorders.

### RELATED ART

Headache disorders are among the most disabling health conditions worldwide. While about 50% of the world population between the age 18 and 65 have had at least one episode of headache in the last year, 30% of this population is suffering from migraine disease, with migraine being the third most common disease worldwide affecting 1 in 7 people. About 10% of the pediatric population experiences migraine. 1.7 to 4% of the world population is suffering from chronic disabling headaches that are affecting half of the time their lives, i.e. more than 15 days/month of headaches. (WHO fact sheet on headache disorders; https://www.who.int/news-room/fact-sheets/detail/headache-disorders, accessed on 23 May 2022).

Headache disorders, including migraine headaches, chronic and episodic cluster headaches and neuralgias, like occipital and trigeminal neuralgia, are severely handicapping conditions whose treatment remains a challenge. For a patient suffering from headache disorders accompanied by skin hypersensitivity normal life is disrupted. Minimal thermal variations, such as exposure to cold or to wind on the head, or sympathetic arousal during the day or night triggers crisis with handicapping pain.

Several therapeutic approaches have been proposed, mainly based on medication, which range from abortive headache drugs to alleviate pain during crisis or disease modulating drugs to reduce the general burden of the disease. Non-drug based approaches are aimed to ablate or stimulate main peripheral nerve trunks responsible for carrying sensation and pain in the head and neck.

The limitation of drug-based treatments are: the limited effectiveness for some patients, the side effects that interfere with work ability and normal life style and drug abuse, which may cause medication overuse headaches.

The most prescribed abortive headache drugs are triptans and ergot derivates. These medications are used to decrease pain intensity during crisis in migraine and cluster headache patients. Pain reduction is achieved after 2 hours from intake in 42-76% of patients. Freedom from pain is achieved after 2 hours from intake in only 18-50%. After 24 hours from intake of the drug, the crisis continues in 50% of the patients leading to repeated intake, drug overuse and finally chronic medication induced headaches and escalating side effects. Since triptans are most effective when taken at the early stage of the crisis, sometimes even before the pain starts, fast absorbed and short acting forms such as intra-nasal sprays, subcutaneous injections or sub-lingual forms are preferred for the most severe forms of headaches such as cluster headaches, sometimes requiring multiple doses in a single day in severe cases. If pain intensity decreases within 2 hours after intake at least by half, the drug is considered effective. Side effects, such as thorax pain/pressure, paresthesia, sleepiness, inability to concentrate, nausea and fatigue are common. Abortive drugs may become less effective over time and patients increase the intake approaching or overcoming the recommended safety limits. Other treatments, defined as disease modifying drugs, are meant to reduce the burden of chronic headaches, neuralgias and migraines. These drugs include beta-blockers, anti-epileptics, calcium-antagonists, anti-depressive and anti-CGRP antibodies. These medications when taken regularly may reduce the frequency and severity of the pain suffered by patients. Also, the disease modifying drugs have side effects and may not be sufficient in most of the cases. The wide range of drug types used as migraine prophylaxis suggest that migraine headaches pathophysiology is still poorly understood.

Yet other approaches, for example as described in documents WO2012075209 and EP3681584, use transdermal drug delivery systems with microneedles, for transcutaneous or intracutaneous delivery of anti-migraine agents, such as triptan or sumatriptan, in an area, such as the arm, which is far from the painful zone. Pharmacokinetic issues have raised questions about the safety of this approach.

Alternative approaches to drugs for refractory or intractable migraines have been proposed, such as neurostimulation or radiofrequency/thermal ablation of extracranial nerves, which are invasive strategies with high risk of complications. These approaches target peripheral nerves that are located in the subcutaneous layer or in deeper anatomical structures such as the subfascial or submuscular layers. These nerves are macroscopically visible nerve branches defined by their name. In the case of headaches and neuralgias, the trigeminal nerve and the greater and lesser occipital nerve are the main targets of such treatments.

For these procedures, peripheral nerves are identified with ultrasound or other radiologic investigation or exposed by surgical exploration and are treated by coagulation or stimulation. The most of these procedures such as thermal ablation via cryotherapy, inducing cold, or radiofrequency, inducing heat, of an anatomically identified sensitive nerve branch are temporary, lasting from a few weeks to a few months depending on the method. These approaches carry also the risk of worsening the symptoms due to intrinsic and irreversible nerve lesions. Moreover, these procedures cause temporary high level of pain to the patients that are often difficult to control by pain medications and may last up to several days. As these treatments damage main sensitive nerve branches, these methods lead to a zone of numbness. The main targets in headaches of these approaches are the main sensitive extracranial nerves that are responsible for large cutaneous territories. For example the greater and lesser occipital nerve innervate the posterior hemi-cranial area while the second main target, the supra-orbital nerve innervates the entire front of its side. As a result, the lesion to these main sensitive branches by these methods results in change of sensation to loss of sensation depending on the technology, which may be uncomfortable to the patients. Taken together, patients may abandon these treatment options due to lack of long-term benefits.

Another approach is the intra-muscular injection of neurotoxins, such as botulinum toxin, which have been tested in clinical trials, the so-called Phase 3 Research Evaluating Migraine Prophylaxis Therapy “PREEMPT” trials. High doses of botulinum toxin, for example 165-195 U, and multiple, painful injections, typically 31-39 in one session, were required to achieve improvement of the headaches of the patients in most cases. Botulinum toxin injections are temporary, requiring to be repeated each 2-3 months. Moreover, botulinum toxin injections are known to cause complications, such as pain in the injected site, muscle wasting, dsysphagia, blepharoptosis and diplopia.

There is therefore a need to find a safe way to treat dysfunctions of intracutaneous nerve fibers and/or the neurovascular plexus, or the symptoms of these dysfunctions, without causing severe side effects.

### Short Disclosure of the Invention

The present invention aims to provide a method to configure a medical device or system in such a way that it is suitable for use in the treatment of dysfunctions of the intracutaneous nerve fibers and/or the neurovascular plexus, for example headache disorders.

It is another aim of the present invention to configure a medical device or system such that it can be safely used for treatment of the symptoms of these conditions, without causing severe side effects for the patient.

It is yet another aim of this invention to provide an alternative to existing methods for treating dysfunctions of intracutaneous nerve fibers and/or the neurovascular plexus, which can cause headaches and other disorders.

According to the invention, one or more of these aims are attained by the object of the attached claims, and especially by its independent claims. Further optional embodiments of this invention are provided in the dependent claims.

The invention achieves one or more of its aims by parametrizing medical equipment known in the art. Specifically, the invention achieves one or more of these aims through a method for controlling and/or adapting a microneedling apparatus, having one or more microneedles and means to provide radiofrequency (RF) energy, for treatment of dysfunctions of intracutaneous nerve fibers, comprising the following steps:
setting a defined percutaneous penetration depth for the one or more microneedles ranging from 0.5 mm to 3.5 mm, and
setting an RF energy output value of 5 mJ to 60 mJ, preferably of 10 mJ to 40 mJ, per microneedle.

The defined percutaneous penetration depth is chosen in this range, such that the tips of the one or more microneedles extend into a target area within the dermis layer when passing through the skin of a subject's body part, for example a subject's head.

The term microneedle as used herein means a needle with a portion for inserting into the skin having a maximal diameter of 50 μm to 300 μm, preferably of 100 μm to 250 μm. A microneedling apparatus, unit or device is an apparatus, unit or device comprising microneedles for insertion into the skin.

The microneedling apparatus of this invention is preferably an automated microneedling apparatus. In an automated microneedling apparatus the position of the microneedles and/or a member for supporting one or more microneedles in respect of a treated patient can be set. In particular, the percutaneous penetration depth of the microneedles is configurable in the microneedling apparatus.

Upon receiving the corresponding instruction, the microneedling apparatus causes the microneedle and/or a member supporting one or more microneedles to take a set position.

It is however also possible to manually move a member supporting the one or more microneedles in a desired position. In this embodiment the microneedling apparatus is configured to automatically extend the one or more microneedles such as that their tips reach the pre-defined percutaneous penetration depth.

The automation of the parametrization of the microneedling apparatus allows for precision in the subsequent delivery of the treatment to the patient. The precision is further enhanced by automating the treatment process. Automation also serves to improve reliability of the treatment process.

The parametrization of the microneedling apparatus according to this method minimizes the risk of tissue damage, damage to nerve tissue, in the treated target area. Severe side effects are thereby prevented.

Intracutaneous nerve fibers include pain receptors and fibers, terminal nerve endings, perivascular fibers, small nerve fibers and sympathetic nerve fibers within the skin.

Dysfunctions of intracutaneous nerve fibers and/or blood vessels include trigeminal neuralgia, allodynia, metabolic peripheral neuropathy, such as diabetic neuropathy.

Trigeminal neuralgia includes spontaneous, post traumatic, post-infective, vascular, and post-surgical, also referred to as iatrogenic, trigeminal neuralgia.

Allodynia includes post-traumatic peripheral neuropathy, post-surgical peripheral neuropathy, idiopathic peripheral neuropathy as in migraines, spontaneous allodynia, or allodynia of unknown source.

The dysfunctions of intracutaneous nerve fibers and/or blood vessels may be defined as chronic regional pain syndrome.

Dysfunctions of intracutaneous nerve fibers and/or blood vessels also include pain symptoms associated with autoimmune conditions, spontaneous or iatrogenic neuropathies, such as fibromyalgia, post radiation neuropathy or post chemotherapy neuropathy.

Dysfunctions of intracutaneous nerve fibers further include post herpetic neuralgias, as well as skin hypersensitivities, for example caused by viral infections, such as shingles caused by herpes zoster virus.

Dysfunctions of intracutaneous nerve fibers and/or blood vessels also include alopecia. Alopecia may be androgenetic, areata, fibrosing, diffuse, scarring or universal alopecia.

Dysfunctions of intracutaneous nerve fibers and/or blood vessels also include erectile dysfunction. Erectile dysfunction may be spontaneous, post-traumatic, iatrogenic or from other source.

Peripheral neuropathies may be caused by small fibers or by fibers of the autonomic nervous system.

Dysfunctions of intracutaneous nerve fibers and/or blood vessels include headache disorders.

The term headache disorders as used herein includes common headaches, chronic headaches, allodynia and migraine headaches, cluster headaches, neuralgia crises and/or related cutaneous hypersensitivities in the head. Typical symptoms of these conditions are allodynia, skin hypersensitivity, pain and/or heightened sensitivity to light touch or other sensory stimuli.

In one aspect of this invention the apparatus outputs RF energy pulses having a pulse duration of 1 millisecond to 1000 milliseconds, preferably 100 to 500 milliseconds.

In one aspect of this invention the apparatus outputs RF energy pulses having a pulse intensity of 1 to 50 watts, preferably 5 to 20 Watts.

In one aspect of this invention the temperature of the tissue area targeted with RF energy pulses is heated to a temperature of 40° C. to 90° C., preferably of 60° C. to 80° C.

In one aspect of this invention the apparatus outputs RF energy pulses having a pulse inducing punctual spots of white coagulation of dermal tissues of a diameter of 10 to 500 μm, preferably 100 to 400 μm.

In one aspect of this invention the apparatus outputs RF energy pulses at single or multiple depths of 0.5 to 3.5 mm, preferably 1 to 2 mm. In one aspect of this invention the apparatus outputs RF energy at single or multiple depths of 0.5 to 3.5 mm, preferably 1 to 2 mm.

In one aspect of this invention the apparatus outputs RF energy pulses at a frequency of 200 kHz to 800 kHz, preferably 500 kHz to 700 kHz.

In one aspect of this invention the apparatus delivers a total RF energy of preferably 1 J to 100 J, preferably 15 J to 50 J, per 5 cm² of treated target area in the dermis.

Preferably, RF energy is provided to the target area in form of bipolar RF energy through two or more microneedles.

The microneedles penetrate the dermis layer such as to deliver a defined therapeutic amount of RF energy to or in proximity of small nerve fibers and microscopic blood vessels extending into the dermis layer. The treatment targets primarily the small nerve fibers and the microvascular network in the dermis layer of the skin. Such small nerve fibers may comprise thermal and pain nerve fibers. Small nerve fibers may be autonomic or somatic nerve fibers. Small fibers are also known as small myelinated (Aδ) fibers or unmyelinated C fibers. Other fibers present in the dermal layer are large myelinated Aβ-fibers sending branches to dermal corpuscles and hair follicles. Also, these fibers may be targeted by the RF treatment.

The microneedles can therefore be parametrized for the treatment of small nerve fiber neuropathies or other peripheral neuropathies. However, the parametrized microneedles can also reach other intracutaneous nerve fibers and/or blood vessels. Importantly, the microneedles are parametrized and subsequently applied, such as to avoid complete ablation of the intracutaneous nerve fiber and vessel network during the treatment. Transient decrease of allodynia may be observed after the treatment.

The parametrization of the microneedles may be specifically adapted to a condition. The parametrization may furthermore be specifically adapted to an individual patient and/or the part of the peripheral body part to which the treatment is administered.

The microneedles may be parametrized for the treatment of headache disorders, even though no link between small fiber and/or intracutaneous nerve fibers including intracutaneous sympathetic nerve endings and headache disorders has been made to date. No treatment targeting directly small fibers have been described to date. Targeting the terminal sympathetic and somatic cutaneous branches intracutaneously via delivery of RF energy in order to decrease headache disorders is therefore an entirely new approach proposed by this invention. Small fibers and intracutaneous nerve endings and/or blood vessels are largely interconnected generating a dense network with connections beyond known anatomic distributions of peripheral nerves. This network is primarily located in the dermis layer of the skin. Treatments, such as RF radiation, as previously described, targeting only macroscopical, main peripheral nerves and peripheral vessels fail or only partially succeed, since only a limited number of peripheral nerves are modulated by such a treatment. In contrast, by targeting the network of small nerve fibers in the dermis layer, the treatment is more effective in the target areas comprising blood vessels and in which such nerve fibers are connected among themselves as well as with nerves of the central nervous system.

When extending into the dermis layer, the microneedles may also deliver a defined therapeutic amount of RF energy to the subdermal neurovascular plexus.

To achieve best clinical results, it is important that the tips of the microneedles penetrate into the dermis layer to deliver the RF energy in this layer where the small fibers, pain receptors and terminal somatic and sympathetic nerve endings and the microvascular plexus are mostly concentrated. Delivery of RF energy to the epidermal layer of the skin may be insufficient to modulate the nerve fibers permanently in order to treat the targeted dysfunctions, for example headache disorders, effectively. Moreover, by delivering RF energy into the deeper dermis layer of the skin, superficial skin injury and hyperpigmentation can be avoided.

Current treatment methods of migraines and headache disorders which target the intracutaneous neurovascular structures have so far exhibited only limited results.

For example, treatments based on topical application of therapeutic agents, show less effective due to a limited penetration of the agent through the skin layers. Moreover, topical applications usually have a relatively slow absorption rate, such that the symptoms can only be alleviated with a significant delay. The method or this invention provides for the RF energy to be delivered directly in the dermis layer and in a target area.

When provided systemically, therapeutic agents require high doses and are required to be taken early at the onset of the crises. When taken as disease modifying therapeutic these agents must be administered in a continuous and regular manner, resulting in an increased risk of side effects and drug abuse or addictive behavior. Whilst the described method may be used for repeated and even regular treatment, depending on the type and severity of the headache disorder, The response to the treatment, the above-mentioned risk of drug-related side effects and addictive behavior are avoided.

Repeat and/or regular treatments of a patient with an apparatus parametrized according to this method may be desirable or necessary to achieve long-term effectiveness of the therapy. However, depending on the type of neuropathic dysfunction, its severity, and the specific case, a single treatment may also prove efficient in the long term. The frequency of administering such treatments and the number of treatments to be provided may be customized on a case-by-case basis.

The described method parametrizes the microneedling apparatus such that the tips of the microneedles provide the adequate therapeutic RF energy in close proximity to and/or directly to the small nerve fibers, pain receptors, terminal somatic and sympathetic nerve endings, and/or the microvascular plexus in the dermis layer of a patient's body part, in which the symptoms are experienced. Depending on the region of the body part, for example the head, in which the target area is located, the subdermal neurovascular plexus is also targeted by this method. The provided energy causes an inactivation and/or modulation of the disorder, for example a headache disorders.

The RF energy provided to targeted nerve fibers modulates and/or inactivates the small intradermal nerve fibers and microvascular plexus, but does not cause massive damage to peripheral nerves and vessels. Permanent damage of the main nerve fibers is therefore avoided as opposed to the previously described use of RF energy whose main goal is to ablate main nerve fibers.

When RF energy is applied to the dermis layer it meets the resistance of the body tissue resulting in generation of heat energy.

In order to ensure that the target area receives an adequate amount of energy and to control tissue damage due to energy transfer, it is therefore advantageous to measure impedance of the target area. The measurements can be conducted before or during treatment of the target area. An adequate amount of energy is an amount which is sufficient to modulate the targeted nerve fiber network and which does not cause excess, unnecessary damage to surrounding tissue. In continuous measurements a sudden change in impedance of a target area may for example prevent extensive local tissue damage.

Impedance can be determined with microneedles in procedures commonly known in the art. Impedance values can for example be obtained by inserting two or more microneedles into a defined area in the dermis layer of the skin and providing a defined energy pulse through the tips of at least two of the microneedles. The microneedles can be provided with or serve as electrodes to determine the impedance of a defined tissue area.

Further electrophysiological monitoring tools, such as skin galvanic response and electroencephalograms, can be usefully combined with the methods described to optimize the energy delivery and the results.

The obtained impedance values can be used to adjust the parameters of the apparatus, such as RF energy, duration of an individual RF pulse and/or RF frequency energy output.

Impedance may for example be measured during treatment, and/or for each RF pulse. RF output may be adjusted continuously based on the received impedance values. Ideally, impedance values should be received in real-time to allow for adjustment of the parametrization in real time.

Impedance measurements as described above may also be used to define a suitable target area in a subject's skin. To this end, impedance measurements may for example be performed at different depth in the skin.

The adjustments to the parameters of the apparatus, including RF energy, duration of an individual RF pulse and/or RF frequency energy output, may be performed automatically. It is however also possible, that the adjustments are made manually.

If adjustments are based on impedance values and are made manually, the determined impedance values need to be displayed to the user.

Parameters may be set and/or adjusted manually or automatically on the basis of subject-relevant information.

Parameters may be predefined, for example as a set of parameters suitable for treatment of a type of condition, such as for migraine or for a cluster headache, a type of patient, considering for example the age, gender and health of a patient, zone to be treated and/or other types of circumstances. It is possible that predefined parameters or sets of parameters are adjusted during treatment of the patient, for example based on impedance measurements.

Whilst this invention concerns the treatment of dysfunctions of intracutaneous nerve fibers, such as headache disorders, by providing targeted RF energy, the person skilled in the art will understand that a microneedling apparatus can also be parametrized to provide other types of energy to target areas through the tips of the microneedles to target areas in the dermis layer of the skin. Such types of energy may include heat and/or optical energy, such as provided by laser light. For these examples, which are not explicitly claimed by this invention, suitable parameters must be chosen which are sufficient to modulate and/or inactivate dysfunctions, for headache disorders without causing excessive damage or ablation to peripheral nerve fibers and/or the neurovascular plexus. Moreover, for these examples, the apparatus must be equipped with or operatively connected to suitable source of energy, for example heat energy or laser radiation. For these examples, temperature to be reached in the dermis should range between 40° C. to 90° C., possibly distributed in arrays of microscopic areas of about 10 μm to 500 μm in diameter, which are densely distributed at a depth of 1 to 2 mm.

With respect to what is known in the art, the invention provides the advantage that a defined amount of energy for treatment of dysfunctions of intracutaneous nerve fibers, including headache disorders such as migraines, is delivered directly and in a targeted manner to the relevant nerve fibers, such that these conditions are treated efficiently and with minimal side effects. In particular, the method does not cause main peripheral nerve ablation or drug-related side effects.

The here described method permits for the first time to target the intracutaneous microscopic terminal branches of sensitive and autonomic nerve fibers and their microvascular cutaneous plexus. The treatment of these microscopic structures not only allows to target a precise and limited cutaneous patch, which corresponds to the area of maximum pain, but also does not alter sensation neither cause prolonged post-interventional pain. Therefore, the here proposed method permits for a treatment of dysfunctions of intracutaneous nerve fibers including headaches disorders is not only novel with respect to the targeted structures, but also less invasive, less painful and causes less side effects that previously described medical devices targeting large, main cutaneous extracranial nerve branches.

The invention is not particularly limited to any specific type of apparatus. For the delivery of RF-energy the apparatus must include an RF generator.

The apparatus may comprise different subunits, for example a display subunit, a processor subunit, a control unit and a microneedling subunit. An example for a suitable apparatus is described in US 2019262065 A1.

The apparatus may be part of system comprising additional units, such as a processor unit.

The apparatus may also be a handheld microneedling apparatus operatively connected to external units of a microneedling system.

The invention also concerns a computer implementation of the method, a computer program product for execution of the method and a computer-readable data carrier for causing the apparatus to perform the steps of the method.

The invention furthermore concerns an apparatus configured to execute the steps of the method.

Conditional language used herein, such as, among others, “can”, “might”, “may”, “for example”, “such as”, and the like, unless specifically stated otherwise, or otherwise understood within the context as used, is generally intended to convey that certain embodiments include, while other embodiments do not include, certain features, conditions, states, parts, and so forth.

The terms “comprising,” “including,” “having,” and the like are synonymous and are used inclusively, in an open-ended fashion, and do not exclude additional features, conditions, states, parts, and so forth.

### SHORT DESCRIPTION OF THE DRAWINGS

Exemplar embodiments of the invention are disclosed in the description and illustrated by the drawings in which:

FIG. 1 is a schematic presentation of sensitive territories of the human head and neck, where possible treatment targets are situated. The frontal or supra-orbital I, the infraorbital II, the temporal and mandibular III, the occipital IV and the cervical, auricular and submandibular V regions are illustrated.

FIG. 2 is a schematic presentation of the microneedling subunit of the apparatus with a plurality of microneedles (2) penetrating into the dermis layer (D) of the human skin.

FIG. 3 illustrates schematically the epidermis E and dermis D of the skin with small nerve fibers F and the neurovascular plexus NVP. The main target of the treatment is indicated by the symbol ⊕ (A).

### EXAMPLES OF EMBODIMENTS OF THE PRESENT INVENTION

The invention provides a method to parametrize a medical device to modulate and/or alleviate dysfunctions of intracutaneous nerve fibers including headache disorders, such as migraines, chronic headaches, cluster headaches, neuralgias such as occipital neuralgia, allodynia or skin hypersensitivity pain in the head.

To provide a targeted treatment of the disorder, the patient points out a distinct area or areas of origin of pain, or most painful point(s) in a body part, for example in the head and neck. Preferably the area of pain origin identified by the patient should not exceed 5cm² , or 3 cm² , or 1 cm². It is important to distinguish between the area of the origin of pain, which is usually a well-defined point or several points of the size of an average tip of an index finger, depending on the crisis, and the region in which the pain spreads. Due to the dense interconnections between small nerve fibers this region may be quite large and include several peripheral nerve territories.

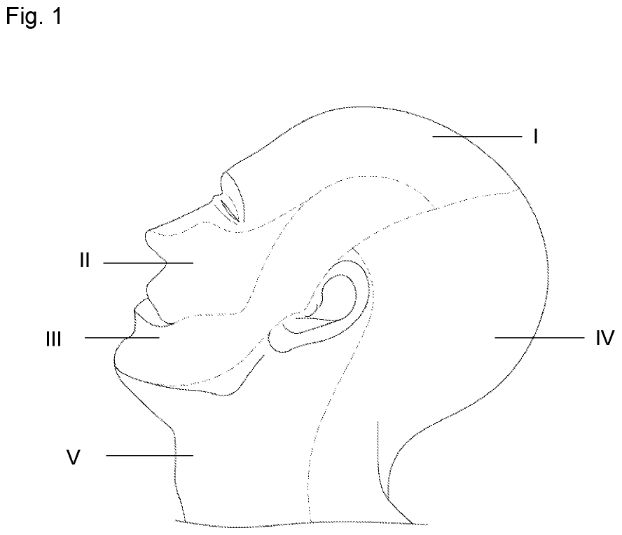

In most cases, the areas in which the pain is felt are primarily located in the frontal region I, extending along the supra-orbital, supra-trochlear nerve territories, in the temporal region III, covering the area along the course of the superficial temporal artery and auriculo-temporal nerve, and/or occipital region IV, which covers the areas of the greater and lesser occipital nerves. Pain may however also be located in any region of the head and neck. The different regions of the head are depicted schematically in FIG. 1.

Pain may however also be located in other parts of a patient's body, respectively in specified regions of said body parts.

The area identified by the patient may be marked, for example with a skin marker or any other marking tool. The area may be anesthetized, for example with a topical local anesthetic cream and occlusive dressing, or with a local anesthetic injection or nerve block. In preparation for treatment the skin in the area is cleaned and prepped with disinfectant and dried.

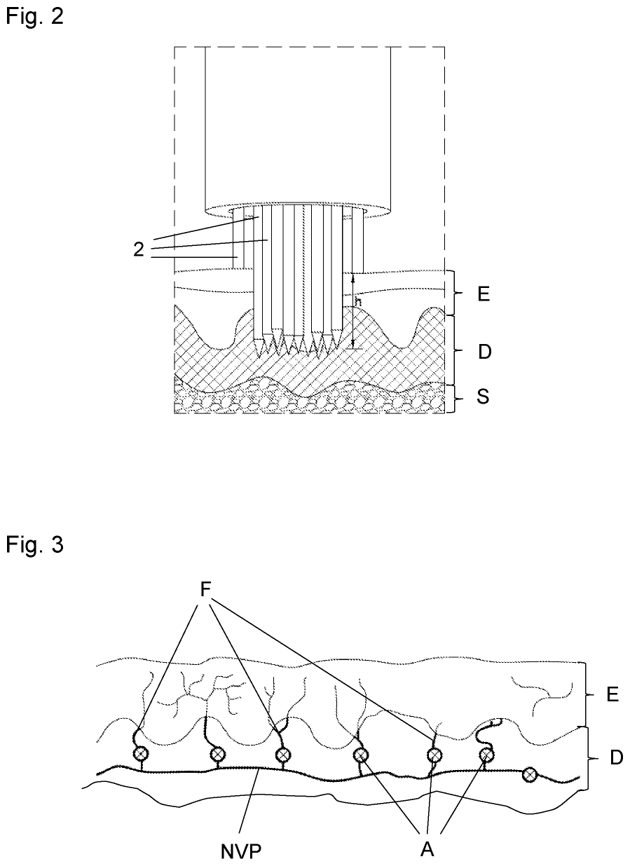

A microneedling apparatus suitable for inserting one or, a plurality of microneedles, preferably 2 to 100 microneedles, which are arranged with a distance of 0.1 to 1 mm between individual microneedles, is configured to permit for an insertion of microneedles into the skin of the patient up to a defined depth h (FIG. 2). The defined depth is a value between 0.5 mm and 3.5 mm, which corresponds to a depth reaching the dermis layer in the skin of the human head and body. The actual penetration depth may be chosen within this range according to the location of the area of pain pointed out by the patient, based on typical depth of the dermal layer in this region of the head, or of another body part as the case may be, and, optionally, accounting for expected physical variations of the patient.

FIG. 2 depicts the microneedling subunit of an apparatus, wherein the microneedles are inserted into the skin such that the tips of the microneedles extend into the dermis layer. The epidermis E, dermis D and subcutaneous tissue S are indicated in this illustration.

A target area for treatment in the dermis layer can be defined based on the surface area pointed out by the patient and on the expected depth of the dermis layer at this point.

The target area is preferably accessed with microneedles, FIG. 2, that have an isolated shaft and transmit energy only though the tip at a desired depth to avoid unnecessary epidermal and dermal destruction.

The microneedling subunit of the apparatus may be a hand-held unit. The microneedling subunit is positioned in contact or at a defined distance from the surface of the area identified by the patient. The microneedles are then inserted into the skin at the set depth h.

The microneedles of the microneedling subunit may be in a retracted position, wherein the tips of the microneedles 2 do not extent beyond the microneedling subunit's surface area. This surface area is the positioned to interface with the skin surface area identified by the patient. Upon activation, for example by a click of a button on the microneedling subunit or a pedal, the microneedles are released from their retracted position and into an extended position to penetrate the skin to the set the depth h according to this invention.

In one embodiment of this invention the microneedling apparatus is then set to provide RF energy of 5 mJ to 60 mJ per needle, preferably of 10 mJ to 40 mJ per needle through each of the inserted microneedles to the target area in the dermis layer. The radiation frequency is preferably set to a value from 500 kHz to 700 kHz.

In a preferred aspect RF energy is provided in pulses, wherein a preferred pulse duration of 1 millisecond to 1 second is chosen.

For example, RF energy output may be set to 20 mJ per needle at a frequency of 600 kHz and provided in pulses of 100 to 500 milliseconds.

In order to cover a wider area multiple RF radiation passages, preferably up to three, are performed. The total energy delivered to a target area of approximately 5 cm² is preferably 15 J to 100 J. The total energy delivered to a target area may be adjusted according to the region of the body part, for example the region of the head, in which the target area is located and/or the characteristics of the skin in this area. For example, in the temporal region, where the skin is particularly thin, lower energy may be required, while in the occipital region, where the scalp skin is thicker and dense in hair follicles, higher energy may be chosen.

Following the treatment, no dressing is required to cover the insertion sites. The patient may immediately return to his/her normal activities.

FIG. 3 depicts the epidermal and dermal skin layers with the main targets A for energy treatment indicated by the symbol ⊕. This figure shows terminal sensitive cutaneous nerve branches, including somatic, such as temperature-sensing, fibers and autonomic, such as nociceptive or sympathetic, fibers. These fibers are also called small myelinated Aδ fibers and unmyelinated C fibers. These small nerve fibers are found in the dermis and penetrate the epidermis with different patterns. As mentioned earlier, the method disclosed here targets small nerve fibers F in the dermis layer. Depending on the position of the target area, the method also targets the neurovascular plexus NVP in the dermal or subdermal layer.

To avoid overheating of the target area due to a higher than expected resistance of the tissue in the target area, the parameters for RF radiation may be adjusted according to values obtained from impedance measurements in this area. Impedance may be measures through microneedles extending to the target area. Alternatively or additionally impedance may be measured before or during treatment. Adjustments to the settings of the apparatus, including RF energy, pulse duration and RF frequency, may be made before and/or during a treatment session.

Impedance may be measured at different depth in the skin of an area pointed out as an area of source of pain by the patient prior to treatment in order to determine a suitable depth for a target area.

In one aspect of the invention the received impedance measurements are used to set or adjust different parameters of the microneedling device for the treatment.

In one aspect of the invention the microneedling apparatus is provided with user interface for receiving user input data. The user interface preferably includes or is connected to input means, for example a touchscreen, or a keyboard or scroll button, for inputting the desired values of the parameters.

The user interface may also be controlled remotely. The input means may for example be provided as part of an external computer device, such as a tablet or smartphone, which is operatively connected with the apparatus.

The method of this invention may comprise providing a computer processor which is communicatively connected to the microneedling apparatus, with input information.

The processor may be comprised in the microneedling apparatus.

The input information may be a defined percutaneous penetration depth for the one or more microneedles, and a defined an RF energy output value.

In addition or alternatively, such input information may relate to patient-specific information on the basis of which a suitable percutaneous penetration depth and an RF energy output value can be determined by said processor. The input information is used to configure the processor to instruct the microneedling apparatus to position the microneedles at the defined penetration depth and to apply the defined RF energy through the microneedles.

An input means for setting RF-related parameters, such as energy, pulse duration and or duration of a treatment period, may for example be incorporated in a main unit of the apparatus. Another input means for setting the penetration depth of the microneedles may be provided on a handheld microneedling subunit of the apparatus. Several input means may be provided in one apparatus, for example on different subunits of the apparatus.

It is also possible to set the penetration depth mechanically, without the use of a processor, on a microneedling subunit of a device.

For example, in embodiments in which microneedles are reversibly moved between a first retracted position and a second extended position, the positions may be set with a regulating mechanism integrated into the microneedling subunit. The regulating mechanism may for example comprise mechanically fixing a blocking element in a defined position, such that the microneedles can only extend to a set maximum length, corresponding to the desired penetration depth h.

The needles can also be fixed in length to a desired penetration depth between 0.5 mm to 3.5 mm and inserted manually before each treatment period.

Retractable microneedles may also be adjusted such that they project from the device surface at a desired length and may then be fixed in this position, such that the microneedles are inserted to the set depth h when the skin of the patient touches the surface of the device from which the microneedles project.

Alternatively, blocking element fixed at a defined height of the microneedles may be used to set the penetration depth. The blocking element prevents non-retractable microneedles from penetrating further than the desired depth h.

All or some of the input means are preferably connected to a control means, for example a central processor, which adjusts the output values according to the data received through the user interface. The control means may also adjust output values according to data pertaining to measurements performed on the patient, for example impedance measurements.

The control means may adjust the RF generator to output the input energy value. The control means may furthermore adjust the projection mechanism of a microneedling subunit to allow for penetration of the microneedles to a defined depth.

The input values, which may be received by the control means are RF energy values of 5 mJ to 60 mJ per microneedle, preferably of 10 mJ to 40 mJ per microneedle.

The input values may also concern the RF frequency, which is preferably set at 200 kHz to 800 kHz, preferably at 500 kHz to 700 kHz.

The input values may concern the pulse duration of the RF radiation, which is preferably set to 1 millisecond to 1000 milliseconds, or to preferably 100 to 500 milliseconds.

In one embodiment of the invention, the input data relate to the patient-specific information, for example type of headache disorder, age, gender, skin type of the patient, location of the source of pain on the head. In this embodiment, a suitable parametrization of the microneedling apparatus may be defined by the control means on the basis of the patient-and/or condition-specific input data. The control means may be pre-programmed with a defined set of parameters corresponding for example to a predefined headache condition, to a type of patient, and/or to the physical properties of a patient.

A processor subunit of the apparatus or an external processor unit may be receiving data concerning a treatment session of the patient, preferably in a real-time feed-back loop. These data may for example be impedance data received from impedance measurements conducted in the target area in the dermis-layer of a patient's body part, for example the head.

The processor subunit or the external processor unit may be connected to a display unit for presenting information provided by the processor. Such information may concern impedance and other treatment-relevant data. Such information may also comprise suggestions for adaptation of the parametrization based on the received data. The user may adjust the set parameters manually according to the displayed information.

The processor subunit or the external processor unit may be configured to adjust the set parameters automatically according to the received feed-back data concerning the treatment, for example impedance data. Preferably the automatic adjustment is made in real-time.

One treatment session with a defined number of pulses may suffice to alleviate the symptoms of headache disorders on a long-term basis. It is however advisable to monitor symptoms of the treated dysfunction, for example a headache disorder, including their frequency, duration and intensity, in combination with medication intake, if any, for a one to twelve month period after the RF treatment and to compare them with the symptoms experienced prior to RF treatment.

If needed RF treatment may be provided repeatedly or even regularly in a series of treatment cycles. In particular, the procedure may be repeated if only partial improvement of symptoms are observed and/or if new areas for source of pain are identified by the patient.

For some headache disorder conditions a single treatment, i.e. a single period of delivering pulsed RF to the target area in the dermis layer, may be sufficient to permanently alleviate the symptoms of the headache disorder.

### Example 1

### Diverse Headache Disorders

The method provided herein may be used for parametrization of a microneedling RF apparatus to temporarily or permanently inactivate headache disorders.

The method has been used for treatment of over 30 patients suffering from different types of headache disorders with different etiology.

All patients treated with a microneedling apparatus parametrized according to the described method experienced some level of improvement of their condition. No patient reported worsening. No complication other than a local redness and swelling for less than 24 hours was reported.

Specifically, in 45% of the patients the headache disorder went into full remission, 30% patients experienced an estimated 50% improvement and 25% of the patients reported an estimated improvement of less than 50% of their condition following a single treatment.

Improvement is calculated as a combination of headache intensity, frequency and duration, also called migraine headache index, and as a factor of reduced drug intake.

The different types of headache disorders treated were active cluster headaches, chronic headaches, migraines with or without aura, occipital neuralgia, trigeminal neuralgia, postherpetic neuralgia, post traumatic peripheral neuropathy causing permanent or episodic skin allodynia, which is commonly present in migraines.

Of the ten patients treated for active cluster headaches, five were suffering from chronic cluster pain, while five patient experienced episodic clusters of pain. In 90% of the cases, an average of 2.1 treatments with the parametrized microneedling radiofrequency apparatus stopped the symptoms of the condition within 48 h from end of the treatment.

Of the four patients with chronic headaches two patients experienced 100% remission of the symptoms. The two other patients reported an improvement of about 50% of their symptoms.

In the three patients treated for postherpetic neuralgia a partial improvement of about 50% of their burning and itching symptoms was observed following one to three treatments.

Of the 10 patients with post traumatic peripheral neuropathy symptoms, two patients experienced a complete remission, while four patients reported a stable 50% improvement of the symptoms, and four patients reported a less than 50% improvement after a single treatment.

The results provide a clear evidence of the effectiveness of the described invention for the treatment of headache disorders. As mentioned above, a series of treatment cycles may be repeated in the course of treatment of the headache disorder depending on the individual needs of the patient.

To further verify the efficacy of the parametrized system for the treatment of patients suffering from specific conditions, further treatment series were conducted

### Example 2

### Cluster Headaches

Ten patients suffering from cluster headaches underwent micro-radiofrequency treatment using a system according to this invention to test the efficacy of this treatment.

All patients suffered from active cluster headaches, wherein five patients experienced chronic pain and five patients reported episodic symptoms. The patients'average age was 42.6 years, eight male and two female patients were included in the pilot trial.

Seven patients suffered from cluster headache alone, three patients had also other overlapping forms of headaches, such as occipital neuralgia, migraines, post-traumatic chronic headaches.

All patients had a formal diagnosis of cluster headaches from a neurologist and tried most of the available drugs for treating this disease without reaching a level of benefit compatible with a normal life.

The condition of the patients was followed for 3 to 11 months after treatment.

After an average of 2.1 treatments, 90% of the patients responded positively, i.e. with complete remission of the cluster crisis. The number of attacks per day passed from 2.7 to 0.3, corresponding to a 9-fold decrease with p<0.01.

The intensity of the crisis, judged by all the patients who had indicated 10/10 on the visual analog scale before treatment, decreased to an average of 1.1, corresponding to a 9.1-fold decrease, with p<0.01.

The number of drugs taken daily for the crisis, which were mainly injectable and spray triptans, was reduced from 2.2 to 0.3 per day, corresponding to a 7.3-fold decrease, with p<0.01.

The Midas score passed from severe disability, i.e. grade IV in all patients, before the treatment to grade I indicating little to no disability, with p<0.01.

No Complications were observed. Local redness due to the treatment faded in a few hours without treatment.

### Example 3

### Migraine Headaches

To test the efficacy of the parametrized system for treatment of migraine headaches, we performed a retrospective analysis on 20 patients treated.

These patients were suffering from intractable migraine headaches, who already failed to respond to commonly available drugs, infiltrations and surgical approaches.

Eight out of 15 patients, i.e. 53.3%, exhibited over 80% improvement in the migraine headache index, which is calculated as (pain intensity x duration x frequency) after one to three treatments.

Three out of 15 patients, i.e. 20%, exhibited about 50% improvement. Four patients out of 15, i.e. 26.7%, did not show any significant improvement. No patient showed worsening of the headaches.

### Example 4

### Peripheral Neuropathy (Post Traumatic Peripheral Neuropathy with Possible Neuroma Formation)

To test the efficacy of the described parametrized system for treatment of peripheral neuropathy, a retrospective analysis on 10 patients treated with the described method and suffering from allodynia was performed.

Patients developed allodynia from trauma and surgery, for example vascular surgery, hallux valgus, knee arthroplasty and/or other trauma surgery.

Two out of ten patients experienced full remission of symptoms. Four patients reported an alleviation in localized pain symptoms which was superior to 50%. This improvement was stable after one to three treatments. Four patients reported only a minimal improvement, inferior to 50%.

Among the patients reporting full remission one suffered from saphenous nerve allodynia after several vascular surgeries and the other allodynia after hallux valgus surgery. Both pain conditions were possibly related to the formation of post-traumatic neuromas.

Four patients reporting over 50% improvement suffered from post-traumatic chronic pain and allodynia, and were already diagnosed with intractable chronic pain. The neuropathic pain may have been related to nerve lesions post trauma and surgery and formation of a neuroma.

Four patients, i.e. 40% of the treated individuals, did not show any improvement. These patients did not report any worsening of symptoms.

All cases of neuropathic pain, possibly related to lesions of main nerve trunks, would have been candidate for more invasive procedures such as surgery to remove the neuromas, radiofrequency ablation of a main nerve trunk or implantation of a neurostimulator. It is clear that a lesser invasive procedure such as the one described in this invention is useful in the management of complicated post traumatic neuropathies.

### Example 5

### Peripheral Neuropathy Due to Metabolic Disease (Diabetic Neuropathy)

One patient suffered from diabetic neuropathy considered untreatable and had already undergone local infiltrations and peripheral nerve decompression surgery, which did not result in any noticeable improvement. This patient suffered from allodynia of the left leg in a stocking distribution. After two treatments the patient reported an almost complete remission of pain, reaching a level of comfort the patient had not experienced for several years.

### Example 6

### Alopecia

Three patients suffering from androgenic alopecia were subjected to treatment using the described parametrized system. A few weeks after the treatment a subjective improvement in hair density was noted.

### Example 7

### Erectile Dysfunction (ED)

One patient suffering from post-traumatic (post-surgical or iatrogenic) ED reported improvement in function after one session using the described parametrized system.

1.-21. (canceled)

22. A microneedling apparatus for use in the treatment of dysfunctions of intracutaneous nerve fibers and/or the neurovascular plexus comprising: one or more microneedles (2) suitable for percutaneous penetration of the skin to a depth of 0.5 mm to 3.5 mm; and means to provide radiofrequency (RF) energy suitable for providing 5 mJ to 60 mJ per microneedle.

23. The apparatus of claim 22, wherein the apparatus is configured for applying RF energy of 5 mJ to 60 mJ per microneedle (2), to a target area within the dermis layer (D) of the skin through the one or more microneedles (2).

24. The apparatus of claim 23, wherein the apparatus is configured for applying RF of 10 mJ to 40 mJ per microneedle (2).

25. The apparatus of claim 23, wherein the apparatus is configured for providing RF energy pulses having a pulse duration of 1 millisecond to 1000 milliseconds.

26. The apparatus of claim 25, wherein the apparatus is configured for providing RF energy pulses having a pulse duration of 100 milliseconds to 500 milliseconds.

27. The apparatus of claim 23, wherein the apparatus is configured to provide RF energy pulses at a frequency of 200 kHz to 800 kHz.

28. The apparatus of claim 27, wherein the apparatus is configured to provide RF energy pulses at a frequency of 500 kHz to 700 kHz.

29. The apparatus of claim 23, wherein the apparatus is configured to provide a total RF energy of preferably 1 J to 100 J per 5 cm² of target area in the dermis of the skin.

30. The apparatus of claim 29, wherein the apparatus is configured to provide a total RF energy of 15 J to 50 J per 5 cm² of target area in the dermis of the skin.

31. The apparatus of claim 22, wherein the apparatus is configured to provide RF energy exclusively through the tips of the one or more microneedles (2).

32. The apparatus of claim 22, comprising a plurality of microneedles (2) for delivering bipolar RF to a target area in the dermis of the skin.

33. The apparatus of claim 32, the plurality of microneedles (2) comprising 2 to 100 microneedles.

34. The apparatus of claim 22, wherein the apparatus further comprises a processor and/or is communicatively connected to a processor.

35. The apparatus of claim 22, wherein the apparatus is configured for receiving impedance values for a target area in the dermis; and for automatically regulating provided RF energy to adjust RF energy provided to said target area according to a measured impedance value for said target area.

36. The apparatus of claim 35, wherein the apparatus is configured for receiving impedance measurements in real time and for regulating the provided RF energy automatically in real time.

37. The apparatus of claim 22, wherein the apparatus is configured for receiving patient-relevant input information; and for a member selected from setting and/or adjusting penetration depths of the one or more microneedles, RF energy provided, RF pulse duration, and/or RF frequency on the basis of said patient-relevant information.

38. The apparatus of claim 22, wherein the apparatus is adapted to be pre-programmed with a defined provided RF energy, RF pulse duration, and/or RF frequency on the basis of a predefined condition.

39. The apparatus of claim 38, wherein the predefined condition is selected from a predefined headache condition, and/or one or more predefined physical property of the patient.

40. The apparatus of claim 22, wherein the dysfunction of intracutaneous nerve fibers and/or the neurovascular plexus is selected from a group consisting of headache disorders, trigeminal neuralgia, allodynia, a metabolic neuropathy, an autoimmune neuropathy, a spontaneous neuropathy, an iatrogenic neuropathy, alopecia, erectile dysfunction, and a dysfunction associated with a viral infection.

41. A method for controlling and/or adapting the microneedling apparatus of claim 22, comprising: setting a defined percutaneous penetration depth for the one or more microneedles ranging from 0.5 mm to 3.5 mm; and setting an RF energy output value for applying RF energy through the one or more microneedles of 5 mJ to 60 mJ per microneedle.

42. The method of claim 41, further comprising: receiving impedance values for a target area within the dermis; and parametrizing the apparatus to suggest a value of RF energy output, or to automatically regulate RF energy output, such as to adjust RF energy delivered to the target area according to a measured impedance value for said area.

43. A computer program product which contains instructions for controlling and/or adapting the microneedling apparatus of claim 22 and which, when loaded on a computerized system or on a processor of said apparatus, causes the apparatus to perform the steps of: setting a defined percutaneous penetration depth for the one or more microneedles ranging from 0.5 mm to 3.5 mm; and setting an RF energy output value for applying RF energy through the one or more microneedles of 5 mJ to 60 mJ per microneedle.

44. A computer-readable data carrier comprising instructions which, when executed by an external processor or by a processor of the apparatus of claim 22, cause the apparatus to carry out the steps of: setting a defined percutaneous penetration depth for the one or more microneedles (2) ranging from 0.5 mm to 3.5 mm; and setting an RF energy output value for applying RF energy through the one or more microneedles (2) of 5 mJ to 60 mJ per microneedle.
